(19) United States
(12) Patent Application Publication
(10) Pub. No.: US 2024/0085322 A1
(43) Pub. Date: Mar. 14, 2024

(54) OPTICAL SYSTEM FOR TRIGLYCERIDE INSPECTION

(71) Applicant: Taiwan RedEye Biomedical Inc., Hsinchu City (TW)

(72) Inventors: Shuo-Ting Yan, Hsinchu City (TW); I-Hua Wang, Hsinchu City (TW); Chen-Chung Chang, Hsinchu City (TW)

(73) Assignee: Taiwan RedEye Biomedical Inc., Hsinchu City (TW)

(21) Appl. No.: 17/940,068

(22) Filed: Sep. 8, 2022

(51) Int. Cl. G01N 21/53; A61B 5/00; A61B 5/145; A61B 5/1455; A61B 5/1495; E03D 11/00

(52) U.S. Cl. CPC G01N 21/53; A61B 5/0004; A61B 5/14546; A61B 5/1455; A61B 5/1495; A61B 5/6891; E03D 11/00; A61B 2562/0238; G01N 2201/12746

(57) ABSTRACT

The present invention relates to an optical system for triglyceride inspection partially integrated into a toilet seat and comprising a plurality of optical sensor modules and a controlling and processing module, wherein each said optical sensor module comprises a first light source, a second light source and an optical sensor. The optical sensor receives light signals generated by the first and second light sources respectively on the skin of the person (especially the skin of the thighs) to be tested and thereby generates a sensing signal of an adaptive calibration function. The sensing signal is then converted by the controlling and processing module into an inspection value of triglyceride, which is transmitted to a display unit. With the above optical system for triglyceride inspection, triglycerides can be inspected automatically without invasive blood sampling, making the system a convenient home health monitoring device.

### BACKGROUND OF THE INVENTION

### 1. Field of the Invention

The present invention relates to the technical field of the optical system for triglyceride inspection, and more particularly to an optical system for triglyceride inspection installed on a toilet seat and a toilet containing the system.

### 2. Description of the Prior Art

Triglycerides (TGs, often referred to as neutral fats) are a type of blood fat in the human body. When a person eats, the body converts any calories it does not need to use immediately into triglycerides. The triglycerides are stored in fat cells of the human body. Later, hormones release triglycerides for energy between meals. If the person regularly eats more calories than he burns, particularly from high-carbohydrate foods, then he may have high triglycerides, i.e. hypertriglyceridemia, resulting in visceral fat and subcutaneous fat and leading to obesity, fatty liver, cardiovascular disease, etc. High levels of triglycerides in the blood are usually a high-risk factor for atherosclerosis, cardiovascular disease, and stroke, so triglyceride levels can be used as one of the biochemical indicators to assess the risk of cardiovascular disease.

In general, the normal levels of triglycerides in adults are less than 150 mg/dL, the borderline high levels are 150 to 199 mg/dL, the high-risk levels are 200 to 499 mg/dL, and the very high-risk levels are greater than 500 mg/dL. Usually, the levels of triglycerides can only be obtained by a blood test. For the conventional technique of blood test, an invasive blood sampling is required to perform on the subject after a subject fast for 8 to 10 hours and must be carried out by a professional medical institution for biochemical analysis to obtain the levels of triglycerides. Therefore, the triglyceride test by blood sampling lacks immediacy and cannot monitor the triglyceride levels in the human body in real-time to achieve home health monitoring and to prevent and reduce the incidence of diseases.

Although the current blood sampling for triglycerides test is usually performed when the subject is fasting (on an empty stomach), triglyceride levels usually increase significantly after eating and drinking, and in the case of cardiovascular disease or stroke occurrence, the patient is not always on an empty stomach. Therefore, it is of some medical significance to perform triglyceride tests immediately, randomly, or without restriction to fasting to help prevent and reduce the occurrence of cardiovascular disease

From the above description, the conventional method of blood sampling for triglyceride test is limited by the location and method for implementing the test and cannot monitor the levels of triglycerides in the human body in real-time, so there is still room for improvement. In view of this, inventors of the present application have made great efforts in research and eventually provide the optical system for triglyceride inspection according to the present application.

### SUMMARY OF THE INVENTION

The prime objective of the present invention is to disclose an optical system for triglyceride inspection, which can be partially integrated into a toilet seat, or integrated into a toilet. When a user sits on the toilet seat, a light source generates an emitted light that is projected onto the local skin of the thigh of the user. The emitted light will penetrate part of the skin depth, and a scattered light generated therein will be received by an optical sensor. Then the scattered light received by the optical sensor will be used to analyze the signal strength (levels) of triglycerides. In particular, the skin surface characteristics of different users, such as skin tone, skin roughness, number of hairs, etc. may cause the variation in the degree of absorption of the emitted light and the variation in the degree of direct scattering and may subsequently result in a detection error. In response to the skin surface characteristics of different users, the present invention also performs an adaptive compensation for the skin surface characteristics to improve and enhance the accuracy of triglyceride optical detection. In addition, the optical system for triglyceride inspection according to the present invention is a home health monitoring device that can automatically detect the triglyceride levels of the user in real-time without invasive blood sampling. The optical system for triglyceride inspection according to the present invention can help the user to monitor his or her health condition and then proactively prompt the user to adjust the diet immediately to slow down the increase of triglyceride content in the blood and prevent the occurrence of cardiovascular diseases.

For achieving the prime objective mentioned above, the present invention provides an embodiment of an optical system for triglyceride inspection, which can be partially integrated into a toilet seat and comprises:
a plurality of optical sensor modules arranged in the toilet seat, wherein each said optical sensor module comprises:
   a first light source that generates a first emitted light, wherein the first emitted light generates a first scattered light on the skin surface of a user;
   a second light source that generates a second emitted light, wherein the second emitted light partially penetrates the skin of the user and generates a second scattered light; and
   an optical sensor for receiving the first scattered light and the second scattered light and generating a sensing signal using the first scattered light and the second scattered light;
      and
a controlling and processing module coupled to said plurality of optical sensor modules and comprising a microprocessor and a communication unit;
   wherein the microprocessor is configured to control the first light source to generate the first emitted light and the second light source to generate the second emitted light and is configured to receive the sensing signal generated by the optical sensor and to convert the sensing signal into an inspection value of triglyceride, and finally said inspection value of triglyceride is transmitted to a display unit via the communication unit.

In one embodiment, the first light source is a white light source and the second light source is a near-infrared light source.

In one embodiment, the sensing signal is an adaptive calibration function.

In one embodiment, the adaptive calibration function is described in the following steps:
Step 1: check counts of the reflectance of the first scattered light generated by the white light source, wherein said counts of the reflectance are counted by the optical sensor in the optical system for triglyceride inspection;
Step 2: obtain a constant C as follows:

C=(Counts_white light)/(Counts_baseline)
Counts_baseline means the definition of a general skin condition;
Step 3: check counts of the reflectance of the second scattered light generated by the near-infrared light source, wherein said counts of the reflectance are counted by the optical sensor in the optical system for triglyceride inspection;
Step 4: calculate the internal turbidity Y from step 3, wherein Y is the internal turbidity caused by the triglycerides; and
Step 5: show the result Y′=Y/C
   If C>1, then the skin tone is white or bright; therefore, Y′<Y;
   If C<1, then the skin tone is dark, or the skin is rough or hairy; therefore, Y′>Y.

In one embodiment, the wavelength of the near-infrared light source is between 700 and 2500 nm and preferably between 700 and 1100 nm.

In one embodiment, the controlling and processing module is coupled to said plurality of optical sensor modules by wired transmission. In another embodiment, the controlling and processing module may also be coupled to said plurality of optical sensor modules by wireless transmission.

In one embodiment, the communication unit is an Ethernet interface and communicates with the display unit via a local area network and/or the Internet to transmit said inspection value of triglyceride to the display unit.

In one embodiment, the communication unit is a first wireless signal transmission interface and communicates with a second wireless signal transmission interface of the display unit.

In one embodiment, said first wireless signal transmission interface is a Bluetooth communication interface, a ZigBee communication interface, a Wimax communication interface, an NBIoT communication interface, a LoRA communication interface, a WiFi communication interface, a 4G mobile communication interface, a 5G mobile communication interface or a 6G mobile communication interface.

In one embodiment, the display unit is a smart toilet control panel, a smartphone, a tablet computer, a smartwatch, a smart bracelet, a door phone, a desktop computer, a laptop computer, an all-in-one computer, or a server computer. The inspection value of triglyceride displayed on the above-mentioned electronic or computer devices can be further uploaded to a cloud server for storage and analysis by health care organizations to provide users with appropriate health care advice or take necessary medical measures to prevent or reduce the occurrence of cardiovascular disease.

In another embodiment, the present invention further provides a toilet comprising an optical system for triglyceride inspection as described above.

### BRIEF DESCRIPTION OF THE DRAWINGS

The invention, as well as a preferred mode of use and advantages thereof, will be best understood by referring to the following detailed description of an illustrative embodiment in conjunction with the accompanying drawings, wherein:

FIG. 1 is a perspective view of a toilet containing the optical system for triglyceride inspection according to the present invention;

FIG. 2 is an exploded view of the optical sensor module and the toilet seat of the optical system for triglyceride inspection according to the present invention;

FIG. 3 is a sectional view of the optical sensor module shown in FIG. 1;

FIG. 4 is an experimental and numerical analysis diagram of the comparison between the detection results using the optical method for triglyceride inspection according to the present invention and the examining results using conventional blood sampling;

FIG. 5 is a flow chart of the optical method for triglyceride inspection according to the present invention; and

FIG. 6 is a block diagram of the optical system for triglyceride inspection according to the present invention.

### DETAILED DESCRIPTION OF THE PREFERRED EMBODIMENTS

To better illustrate the advantages of the optical system for triglyceride inspection according to the present invention and its contributions to the art, preferred embodiments of the present invention will be described in detail concerning the attached drawings hereafter.

### First Embodiment

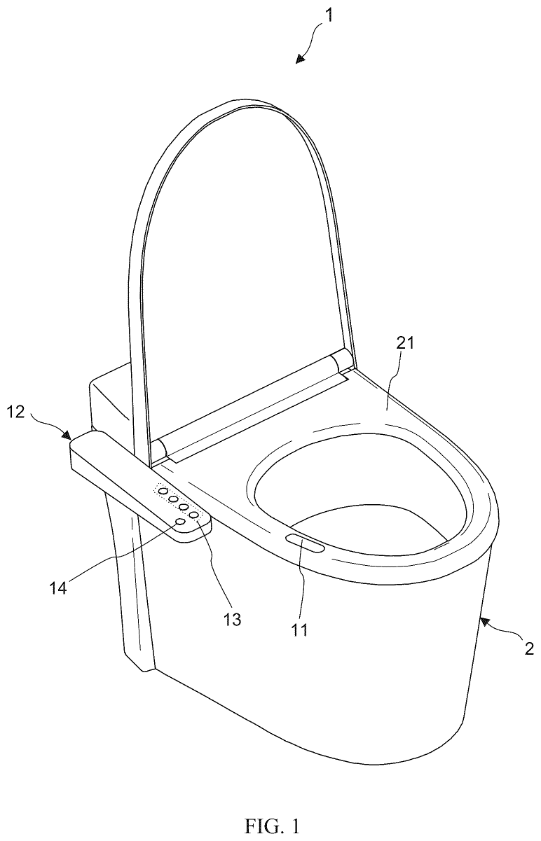

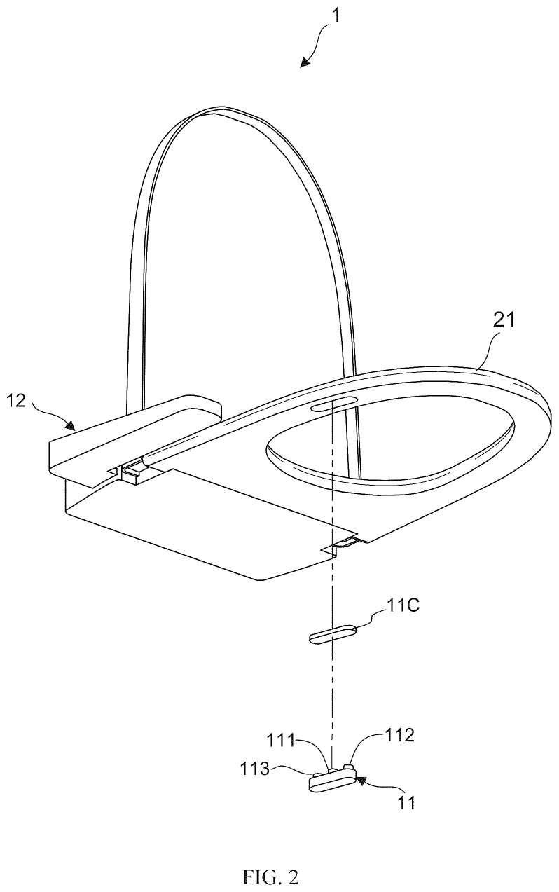

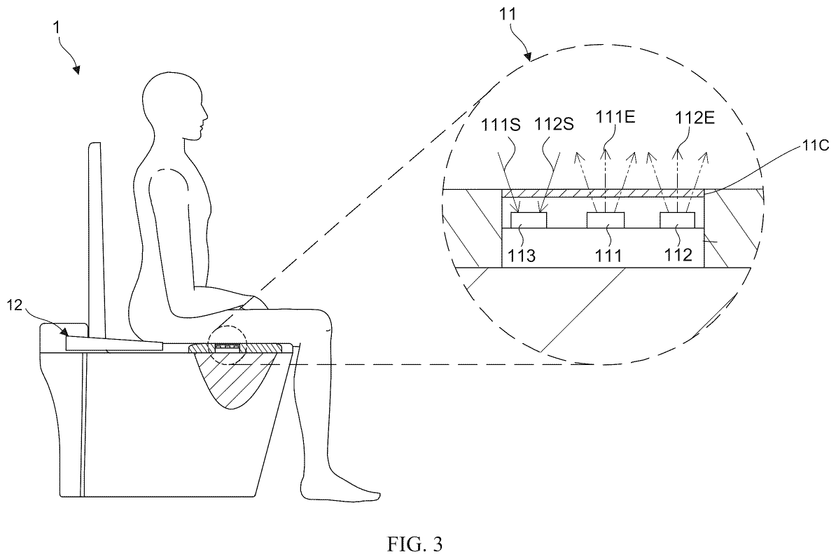

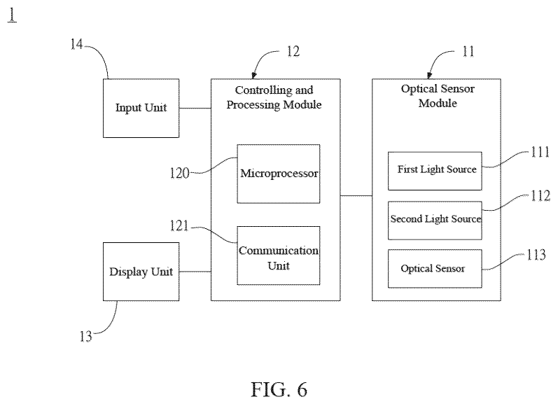

FIG. 1 shows a perspective view of a toilet 2 integrated with the optical system 1 for triglyceride inspection according to the present invention, and FIG. 2 shows an exploded view of the optical sensor module 11 and a toilet seat 21 of the optical system 1 for triglyceride inspection according to the present invention. As shown in FIG. 1 and FIG. 2, the optical system 1 for triglyceride inspection according to the present invention is partially integrated into a toilet seat 21 and comprises: a plurality of optical sensor modules 11 and a controlling and processing module 12 coupled to said plurality of optical sensor modules 11, wherein each of said optical sensor modules 11 is disposed in the toilet seat 21. As shown in FIG. 6, the controlling and processing module 12 comprises a microprocessor 120 and a communication unit 121. Further referring to FIG. 3, each of said optical sensor modules 11 comprises: a first light source 111, a second light source 112, and an optical sensor 113, wherein the first light source 111 generates a first emitted light 111E and the first emitted light 111E produces a first scattered light 111S on the skin surface of a user, wherein the second light source 112 generates a second emitted light 112E, and the second emitted light 112E partially penetrates the skin of the user and produces a second scattered light 112S. In addition, the optical sensor 113 is configured to receive the first scattered light 111S and the second scattered light 112S and to generate a sensing signal using the first scattered light 111S and the second scattered light 112S. Further, the microprocessor 120 is configured to control the first light source 111 to generate the first emitted light 111E, to control the second light source 112 to generate the second emitted light 112E, to receive the sensing signal generated by the optical sensor 113, and to convert the sensing signal into an inspection value of triglyceride. Finally, the inspection value of triglyceride is transmitted to a display unit 13 via the communication unit 121.

In a feasible embodiment according to the optical system 1 for triglyceride inspection, the first light source 111 is a white light source and the second light source 112 is a near-infrared light source. In addition, the wavelength of the near-infrared light source is between 700 and 1100 nm.

Furthermore, in the above embodiment, the sensing signal is an adaptive calibration function, and the adaptive calibration function is described in the following steps:
Step 1: check counts of the reflectance of the first scattered light 111S generated by the white light source, wherein said counts of the reflectance are counted by the optical sensor 113 in the optical system 1 for triglyceride inspection;
Step 2: obtain a constant C as follows:

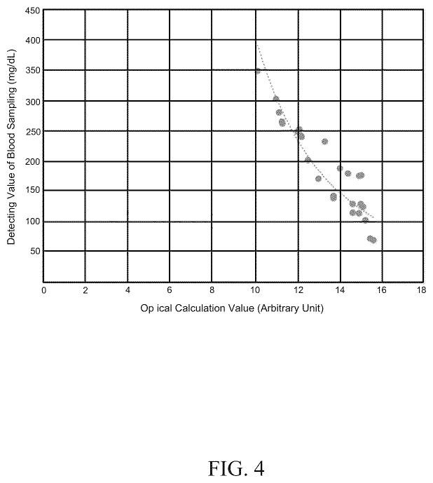

C=(Counts_white light)/(Counts_baseline)
Counts_baseline means the definition of a general skin condition; wherein the Counts_baseline is assumed to be 2670, and if the detection value of Counts_white is 2800, then C=2800/2670=1.05;
Step 3: check counts of the reflectance of the second scattered light 112S generated by the near-infrared light source, wherein said counts of the reflectance are counted by the optical sensor 113 in the optical system 1 for triglyceride inspection;
Step 4: calculate the internal turbidity Y from step 3, wherein Y is the internal turbidity caused by the triglycerides; and
Step 5: show the result Y′=Y/C
   If C>1, then the skin tone is white or bright; therefore, Y′<Y;
   If C<1, then the skin tone is dark, or the skin is rough or hairy; therefore, Y′>Y.
      Besides, C not only represents the feature differences of the skin surface between the users, such as skin tone and hair but also represents the variation caused by the sitting position on the toilet seat. For example, C>1 means that the skin tone is brighter and whiter, and it may also represent that the skin in an inspection area onto which the emitted light is projected is tighter due to the pressure applied on the skin by the surface of the toilet seat. C<1 means that the skin tone is darker or the skin is hairier, and it may also represent that the skin in the inspection area is more relaxed. As shown in FIG. 4, the Y-axis is the detecting value originating from blood sampling (actual value), and the X-axis is the optical calculation value. The dashed line is the result of combining the two axes. The dashed line is an X-Y function, wherein Y is an optical prediction value of internal (blood) turbidity and X is a calculation value of actual optical measurement. The relationship of X and Y in the X-Y function can be exponential, power and polynomial, etc. Through the operation of the X-Y function, the value of internal (blood) turbidity detected by the non-invasive optical system for triglyceride inspection can be obtained. In addition, said function is obtained through induction and summarization of data in the present invention.

In another feasible embodiment according to the optical system 1 for triglyceride inspection, the second light source 112 may further be a green, red or mid-infrared light.

In a further feasible embodiment, as shown in FIG. 2 and FIG. 3, the optical sensor module 11 comprises a transparent cover 11C, wherein the first emitted light 111E and the second emitted light 112E generated respectively by the first light source 111 and the second light source 112 are projected onto the skin of the user through the transparent cover 11C. The first scattered light 111S and the second scattered light 112S are generated respectively after the first emitted light 111E and the second emitted light 112E are projected onto the skin of the user. Then the first scattered light 111S and the second scattered light 112S are received by the optical sensor 113 through the transparent cover 11C. The first emitted light 111E and the second emitted light 112E are projected onto the skin of the user through a part of the transparent cover 11C that has a function of light divergence, such as a diverging lens. The first scattered light 111S and the second scattered light 112S are received by the optical sensor 113 through a part of the transparent cover 11C that has a function of light convergence, such as a condensing lens.

In addition, in the embodiment of the present invention as described above, the communication unit 121 is an Ethernet interface and communicates with the display unit 13 via a local area network and/or the Internet to transmit said inspection value of triglyceride to the display unit 13.

Further, the communication unit 121 is a first wireless signal transmission interface and communicates with a second wireless signal transmission interface of the display unit 13. The first wireless signal transmission interface is a Bluetooth communication interface, a ZigBee communication interface, a Wimax communication interface, an NBIoT communication interface, a LoRA communication interface, a WiFi communication interface, a 4G mobile communication interface, a 5G mobile communication interface or a 6G mobile communication interface.

Moreover, the display unit 13 is a smart toilet control panel, a smartphone, a tablet computer, a smartwatch, a smart bracelet, a door phone, a desktop computer, a laptop computer, an all-in-one computer, or a server computer.

Furthermore, another embodiment of the present invention is a toilet 2 containing an optical system 1 for triglyceride inspection as described above.

### Second Embodiment

The basic principle of the optical system 1 for triglyceride inspection according to the present invention is that when a light source, such as the second light source 112 (LED or laser) as described in the first embodiment, emits a near-infrared (NIR) light beam of a specific wavelength (e.g. 700 to 1100 nm), the NIR light beam penetrates part of the skin depth and produces a scattered light (i.e., the second scattered light 112S in the first embodiment). Then, the scattered light is received by an optical sensor 113, and the scattered light received is used as a basis for analysis of the triglyceride concentration in the human body. In addition, since the penetration depth of different wavelengths of light to skin tissues may vary, near-infrared light (wavelength 700 to 1100 nm), which has better penetration to the skin, is selected as the main light source for the detection of triglyceride in this embodiment.

If the human body contains higher amounts of triglycerides, the optical sensor 113 will receive scattered light with higher intensity; conversely, if the human body contains fewer amounts of triglycerides, the optical sensor 113 will receive scattered light with lower intensity.

However, the skin surface characteristics of different users may vary greatly, and the variation of these skin surface characteristics, including skin tone, skin roughness, and the number of hairs, can cause the variation in the degree of absorption of the emitted light (near-infrared light) and the variation in the degree of direct scattering. Therefore, the inventors of the present application believe that it is necessary to perform an adaptive compensation (offset) of the light signal (scattered light) received by the optical sensor 113 to enhance the accuracy of inspection value of triglyceride.

In the present invention, thus a new light source, such as the first light source 111 (white LED) described in the first embodiment, is added to produce a scattered light (i.e., the first scattered light 111S in the first embodiment) on the skin surface of the user, and the first scattered light 111S is received by the optical sensor 113. Then the second light source 112 (LED or laser) as described in the first embodiment penetrates part of the skin depth with a near-infrared (NIR) light of a specific wavelength (e.g., 700 to 1100 nm) and produces a scattered light (i.e., the second scattered light 112S in the first embodiment), wherein the second scattered light is then received by the optical sensor 113. Next, the optical sensor 113 uses the received light signal (including the first scattered light 111S and the second scattered light 112S) to generate a sensing signal of an adaptive calibration function to compensate for the detection errors resulting from the differences in skin surface characteristics of different users. The adaptive calibration function is as described in the first embodiment. Later, the sensing signal is processed by the microprocessor 120 in the controlling and processing module 12 to determine the signal strength of triglyceride levels of the user or is converted into an inspection value of triglyceride by the microprocessor 120. Finally, the signal strength of triglyceride levels or inspection value of triglyceride is transmitted to the display unit 13 through the communication unit 121.

Referring to FIG. 4, it can be learned from the experimental and numerical analysis that the detection results of the optical system 1 for triglyceride inspection in the second embodiment can have the same effect corresponding to that of the conventional blood sampling. Further, through data collection and analysis, the present invention also establishes an algorithm of a non-invasive optical system for triglyceride inspection with a confidence level of 85% (R2=0.8551).

Further, as shown in Table 1 below, the optical calculation value of the optical system 1 for triglyceride inspection according to the present invention is compensated through the adaptive calibration function for detection error caused by the differences in skin surface characteristics of different users to obtain an inspection value of triglyceride. The inspection value of triglyceride corresponds to the detection value of the blood sampling, i.e., the inspection value of triglyceride is very close to the detection value obtained from blood sampling. Therefore, the results of FIG. 4 and Table 1 show that the inspection value of triglyceride obtained by the optical system 1 for triglyceride inspection according to the present invention can fully reflect the triglyceride concentration in the user's body. In other words, the inspection value of triglyceride is representative and reliable.

TABLE 1

| Optical | The detection | The value obtained by the adaptive |
|---|---|---|
| calculation | values of the blood | calibration function compensating for |
| values | sampling | the skin surface characteristics |
| 14.6 | 129 | 153.9839198 |
| 10.13 | 348 | 346.0126884 |
| 13.715 | 139 | 176.8594308 |
| 11.26 | 265 | 273.7533304 |
| 15.01 | 177 | 144.821724 |
| 13.29 | 232 | 189.6309032 |
| 12.16 | 242 | 230.8814695 |
| 11 | 302 | 288.2917064 |
| 11.13 | 280 | 280.8860413 |
| 12.1 | 252 | 233.4249912 |
| 13.7 | 143 | 177.2886325 |
| 12 | 248 | 237.755453 |
| 11.3 | 262 | 271.611525 |
| 12.2 | 240 | 229.208079 |
| 14.4 | 180 | 158.7610733 |
| 13.7 | 142 | 177.2886325 |
| 15.2 | 103 | 140.8423942 |
| 14.9 | 114 | 147.200526 |
| 14 | 188 | 168.9831289 |
| 14.9 | 176 | 147.200526 |
| 12.5 | 202 | 217.2007235 |
| 13 | 171 | 199.1280409 |
| 15 | 129 | 145.035664 |
| 14.6 | 115 | 153.9839198 |

In addition, in the optical system 1 for triglyceride inspection according to the present invention, the microprocessor 120 in the controlling and processing module 12 converts the sensing signal into an inspection value of triglyceride and transmits the inspection value of triglyceride to the display unit 13 for direct display, and as shown in FIG. 1, the display unit 13 can also display the grade of triglyceride concentration according to the different inspection values of triglyceride by the following light signals:
1. green light: the normal levels, triglycerides <150 mg/dL;
2. yellow light: borderline high levels, triglycerides are between 150 to 199 mg/dL;
3. orange light: high-risk levels, triglycerides are between 200 to 499 mg/dL;
4. red light: very high-risk levels, triglycerides >500 mg/dL.

In other words, the basic principle of the above embodiment uses near-infrared light as the second light source 112 to partially penetrate the skin for detection of triglyceride concentration, but in further embodiments, practically the second light source 112 is not limited to near-infrared light and can be any light that can penetrate part of the skin depth for detection of triglycerides, such as green light (500 to 600 nm), red light (600 to 700 nm), mid-infrared light (˜3 to 8 μm) and other light sources.

### Third Embodiment

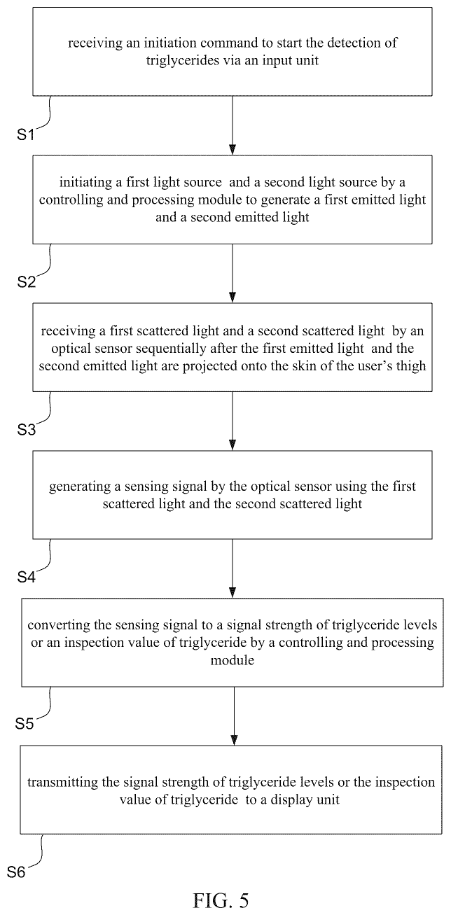

FIG. 5 shows a flow chart of the optical method for triglyceride inspection according to the present invention. As shown in FIG. 5 and FIG. 6, and with further reference to FIG. 3, the method includes the following steps:
S1: receiving an initiation command to start the detection of triglycerides via an input unit 14, i.e., an optical system 1 for triglyceride inspection according to the present invention receives a detection instruction from a user via the input unit 14, wherein the input unit 14 can be a physical button, a voice control module, or an LCD touch display and is coupled to a controlling and processing module 12;
S2: initiating a first light source 111 and a second light source 112 in the optical sensor module 11 sequentially by a controlling and processing module 12 to generate a first emitted light 111E and a second emitted light 112E respectively after the controlling and processing module 12 receiving the detection instruction from the input unit 14, wherein the controlling and processing module 12 is coupled to the optical sensor module 11 and the first light source 111 is a white light LED and the second light source 112 is a near-infrared LED (NIR LED) or a laser;
S3: receiving a first scattered light 111S and a second scattered light 112S sequentially by an optical sensor 113 in the optical sensor module 11, wherein the first scattered light 111S and the second scattered light 112S is generated respectively after the first emitted light 111E and the second emitted light 112E are projected onto the skin of the user's thigh;
S4: generating a sensing signal by the optical sensor 113 using the first scattered light 111S and the second scattered light 112S and transmitting the sensing signal from the optical sensor 113 to the controlling and processing module 12, wherein the sensing signal is an adaptive calibration function according to the present invention as described above and the optical sensor 113 may be a photodiode, a CMOS sensor, a CCD sensor, or a spectrophotometer;
S5: converting the sensing signal to a signal strength of triglyceride levels or an inspection value of triglyceride by a microprocessor 120 in the controlling and processing module 12 to reflect the triglyceride concentration in the user's body, wherein the microprocessor 120 may be an ARM Cortex series microprocessor; and
S6: transmitting the signal strength of triglyceride levels or the inspection value of triglyceride from the microprocessor 120 in the controlling and processing module 12 to a display unit 13 via a communication unit 121, wherein the display unit 13 is coupled to the controlling and processing module 12 and the display unit 13 may be an LED signal light or an LCD touch display.

In a nutshell, the above descriptions have thoroughly introduced the optical system for triglyceride inspection according to the present invention. The above descriptions are made on embodiments of the present invention; however, the embodiments are not intended to limit the scope of the present invention, and all equivalent implementations or alterations within the spirit of the present invention still fall within the scope of the present invention.

What is claimed is:

1. An optical system for triglyceride inspection, which can be partially integrated into a toilet seat and comprises:
a plurality of optical sensor modules arranged in the toilet seat, wherein each of said optical sensor modules comprises:
a first light source that generates a first emitted light, wherein the first emitted light generates a first scattered light on the skin surface of a user;
a second light source that generates a second emitted light, wherein the second emitted light partially penetrates the skin of the user and generates a second scattered light; and
an optical sensor for receiving the first scattered light and the second scattered light, and generating a sensing signal using the first scattered light and the second scattered light;
and
a controlling and processing module coupled to said plurality of optical sensor modules and comprising a microprocessor and a communication unit;
wherein the microprocessor is configured to control the first light source of the optical sensor module to generate the first emitted light and the second light source to generate the second emitted light and is configured to receive the sensing signal generated by the optical sensor and to convert the sensing signal into an inspection value of triglyceride, and finally said inspection value of triglyceride is transmitted to a display unit via the communication unit.

2. The optical system for triglyceride inspection of claim 1, wherein the first light source is a white light source and the second light source is a near-infrared light source.

3. The optical system for triglyceride inspection of claim 2, wherein the sensing signal is an adaptive calibration function.

4. The optical system for triglyceride inspection of claim 3, wherein the adaptive calibration function is described in the following steps:
Step 1: check counts of the reflectance of the first scattered light generated by the white light source, wherein said counts of the reflectance are counted by the optical sensor in the optical system for triglyceride inspection;
Step 2: obtain a constant C as follows:
C=(Counts_white light)/(Counts_baseline)
Counts_baseline means the definition of a general skin condition;
Step 3: check counts of the reflectance of the second scattered light generated by the near-infrared light source, wherein said counts of the reflectance are counted by the optical sensor in the optical system for triglyceride inspection;
Step 4: calculate the internal turbidity Y from step 3, wherein Y is the internal turbidity caused by the triglycerides; and
Step 5: show the result Y′=Y/C
If C>1, then the skin tone is white or bright; therefore, Y′<Y;
If C<1, then the skin tone is dark, or the skin is rough or hairy; therefore, Y′>Y.

5. The optical system for triglyceride inspection of claim 4, wherein the wavelength of the near-infrared light source is between 700 and 2500 nm.

6. The optical system for triglyceride inspection of claim 4, wherein the wavelength of the near-infrared light source is between 700 and 1100 nm.

7. The optical system for triglyceride inspection of claim 1, wherein the second light source is a green light, a red light, or a mid-infrared light.

8. The optical system for triglyceride inspection of claim 1, wherein the communication unit is an Ethernet interface and communicates with the display unit via a local area network and/or the Internet to transmit said inspection value of triglyceride to the display unit.

9. The optical system for triglyceride inspection of claim 1, wherein the communication unit is a first wireless signal transmission interface and communicates with a second wireless signal transmission interface of the display unit.

10. The optical system for triglyceride inspection of claim 9, wherein the first wireless signal transmission interface is a Bluetooth communication interface, a ZigBee communication interface, a Wimax communication interface, an NBIoT communication interface, a LoRA communication interface, a WiFi communication interface, a 4G mobile communication interface, a 5G mobile communication interface, or a 6G mobile communication interface.

11. The optical system for triglyceride inspection of claim 1, wherein the display unit is a smart toilet control panel, a smartphone, a tablet computer, a smartwatch, a smart bracelet, a door phone, a desktop computer, a laptop computer, an all-in-one computer, or a server computer.

12. A toilet comprising the optical system for triglyceride inspection of claim 1.
